(19) United States
(12) Patent Application Publication
(10) Pub. No.: US 2026/0041936 A1
(43) Pub. Date: Feb. 12, 2026

(54) SYSTEMS AND METHODS FOR FLASH THERAPY

(71) Applicant: THE TRUSTEES OF DARTMOUTH COLLEGE, Hanover, NH (US)

(72) Inventors: PETR BRUZA, LEBANON, NH (US); BRIAN POGUE, HANOVER, NH (US); RAMISH ASHRAF, HANOVER, NH (US); RONGXIAO ZHANG, HANOVER, NH (US); DAVID GLADSTONE, NORWICH, VT (US); MEGAN CLARK, Hanover, NH (US); ROMAN VASYLTSIV, Staten Island, NY (US)

(21) Appl. No.: 18/746,659

(22) Filed: Jun. 18, 2024

Related U.S. Application Data
(63) Continuation-in-part of application No. 18/563,813, now Pat. No. 12,036,421.
(60) Provisional application No. 63/193,030, filed on May 25, 2021.
(60) Provisional application No. 63/606,550, filed on Dec. 5, 2023.

(51) Int. Cl. A61N 5/10

(52) U.S. Cl. CPC A61N 5/1065; A61N 5/1031; A61N 5/1049; A61N 5/1077; A61N 2005/1059; A61N 2005/1087; A61N 2005/1089

(57) ABSTRACT

A system provides a pulsed radiation beam along an axis; a scintillator blanket or a scintillator mask conformable to a surface of a target area of a patient; and a camera to image the scintillator from a distance. The system includes an image processor configured to use images of the scintillator to measure radiation dosage provided by each pulse of the beam, totalize radiation dosage provided by pulses of the beam, and provide signals to a controller. A method of monitoring radiation treatment includes placing a scintillator on a target area; providing a radiation beam through the scintillator into the target area; using a camera to image from a distance; and applying dose calibration factors, angular emission correction factors, and a measured 3D surface of the scintillator, to generate maps of dose and dose rate in a geometric plane perpendicular to an axis of the radiation beam.

### CROSS REFERENCE TO RELATED APPLICATIONS

The present application claims priority to U.S. Provisional application 63/606,550 filed 5 Dec. 2023. The present application is also a continuation-in-part of U.S. patent application Ser. No. 18/563,813 filed 22 Nov. 2023. patent application Ser. No. 18/563,813 is a 35 U.S.C. 371 U.S. Entry of PCT/US2022/030837 filed May 25, 2022, and claims priority to PCT/US2022/030837. PCT/US2022/030837 in turn claims priority to U.S. Provisional Patent Application No. 63/193,030 filed May 25, 2021, titled “Systems and Methods for Accurate Flash Therapy.” The entire contents of the aforementioned patent applications are incorporated herein by reference.

### GOVERNMENT RIGHTS

This invention was made with government support under grant no. R01 EB023909 awarded by the National Institutes of Health. The government has certain rights in the invention.

### FIELD

The present document relates to the fields of radiotherapy and dosimetry. In particular, the present application relates to systems and methods that will prevent erroneous delivery of a high-intensity radiation beam.

### BACKGROUND

Radiation therapy (RT), using one or more of x-ray, electron, proton and charged particle beams imparted to patients, has been performed with the beam directionality, energy and cross-sectional spatial intensity modulated to deliver optimized, planned, dose distributions. In order to deliver the planned dose precisely, the beam characteristics and irradiation progress, which typically takes several minutes per treatment fraction to complete, should be monitored in real-time. Recently, radiation delivered at ultrahigh dose rates (UHDR), which are typically 3 to 5 orders of magnitude higher comparing with convectional dose rates over correspondingly shorter treatment times, have demonstrated superior normal tissue sparing than achieved by conventional RT. One of the major challenges is how to monitor and control the irradiation under UHDR conditions since current sensing technologies can become saturated and require using of large correction factors associated with clinically unacceptable levels of uncertainty. More importantly, current validation devices are often not capable of recording dose and dose rate with concurrent spatial and temporal resolution necessary for accurate validation of these UHDR beam sources, such as high-current proton cyclotrons and synchro-cyclotrons, or UHDR electron linear accelerators (LINACs).

UHDR irradiation therapy shows a striking reduction of normal tissue toxicities commonly associated with conventional radiotherapy while also maintaining tumor control, a phenomenon called the FLASH effect. The premise of FLASH or UHDR treatment is to deliver whole dose radiotherapy or fractions at much higher dose rates than with conventional radiotherapy, typically above 40 grays/second (Gy/s) as compared to 0.01 Gy/s in a conventional mode. Ultra-high dose rate irradiation may be performed with electron, proton, or X-ray beams of energies determined to dissipate energy at desired depths in tissue as determined appropriate for individual patients.

There are numerous challenges associated with the rapid nature of FLASH delivery, particularly regarding dose feedback systems and interlock circuitry. Current beam monitor dosimeters are either not useful in high dose rate regimes due to strong non-linearity and/or signal to noise issues, they may experience dose-induced damage when subjected to ultra-high dose rate beams, or they are not fast enough to react on a single pulse, millisecond-by-millisecond basis. In addition, while delivering the radiation dose of an entire therapy session with a single fraction lasting less than a second is typically seen as an advantage of FLASH, it imposes new and extreme requirements from the aspects of safety and patient positioning. In standard fractionated delivery, positioning, motion, or anatomy change errors are usually accounted for during planning and they tend to average out over multiple days of treatment. However, a small deviation in patient position in FLASH can have an impact on a patient's health that is orders of magnitude more severe than with conventional radiotherapy.

Prior art systems and methods for ultra-high dose rate irradiation are not capable of reacting to anatomical shifts that may occur anytime between patient alignment and the end of beam delivery. For example, shifts in position due to a non-compliant patient, inadvertent movement, breathing, etc. all may result in suboptimal treatments. Further, in clinical linear accelerators (LINACs), the feedback mechanisms are based on averaged readouts over extended periods of time (for example 50 milliseconds (ms.) in Varian LINACs). This feedback mechanism is too slow for FLASH.

### SUMMARY OF THE EMBODIMENTS

In a first aspect, a system for ultra-high dose rate irradiation includes a radiation source for providing a beam of radiation in a series of pulses; one or more scintillator detectors at an output of the radiation source to measure beam output and symmetry in real-time; and a controller for receiving an input from the one or more scintillator detectors and providing control signals to the radiation source. In embodiments, the scintillator is a semi-flexible blanket formed of multiple scintillator plaques linked together, or a plastic scintillator molded to conform to a particular patient's face or body at a position where the scintillator will intercept a treatment beam.

In a second aspect, a method of ultra-high dose rate irradiation includes providing a series of radiation pulses; measuring the radiation dosage in each pulse with scintillator detectors positioned on a patient; determining a difference in the radiation dosage between a delivered dose and a prescribed dose; and if the difference is smaller than a single pulse radiation dosage, applying a scaling factor to the width or intensity of the following pulse. In embodiments, the scintillator is a semi-flexible blanket formed of multiple scintillator plaques linked together, or a plastic scintillator molded to conform to a particular patient's face or body at a position where the scintillator will intercept a radiation treatment beam.

### BRIEF DESCRIPTION OF THE FIGURES

FIG. 1 is a block diagram of a system for delivering ultra-high dose rate irradiation therapy, in embodiments.

FIG. 2 is a functional block diagram illustrating some of the functions of a dosimetry controller of FIG. 1.

FIG. 3 is a flowchart of a method for delivering ultra-high dose rate irradiation therapy, in embodiments.

FIG. 4 is an illustration of a mesh type semi-flexible scintillator blanket having hexagonal scintillating plaques.

FIG. 5A illustrates an individual hexagonal scintillating plaque in plan view, while FIG. 5B illustrates the hexagonal scintillating plaque of FIG. 5A in cross section.

FIGS. 6A, 6B, and 6C illustrate an alternative scintillating plaque having a triangular shape.

FIGS. 6D, 6E, and 6F illustrates an alternative scintillating plaque having a square shape.

### DETAILED DESCRIPTION

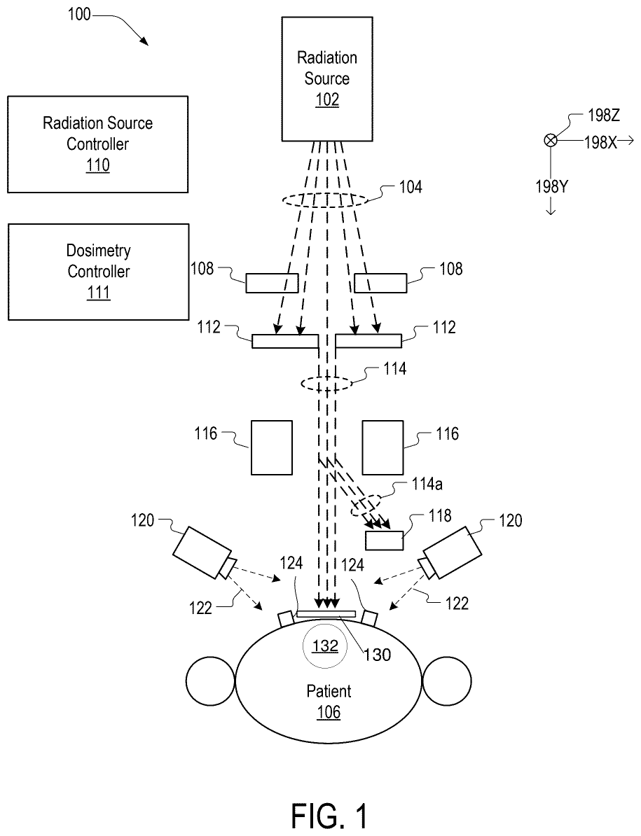

FIG. 1 illustrates a system 100 for delivering ultra-high dose rate UHDR irradiation, or FLASH therapy, to a target area of a patient while preventing erroneous delivery of the radiation beam in terms of total dose and beam position. Systems and methods are described herein in the context of electron beams, but more generally are applicable to all types of charged particle beams such as electrons, protons, heavier charged particles such as alpha particles, and X-ray beams. FIG. 1 includes a coordinate system indicated by x-axis 198X, y-axis 198Y and z-axis 198Z each of which are orthogonal to the others. As used herein a beam axis is parallel to y-axis 198Y and an image target plane is parallel to the x-z plane formed by x-axis 198X and z-axis 198Z.

Radiation source 102 outputs a radiation beam 104 towards a target area in an image target plane of patient 106 along a beam axis parallel to y-axis 198Y. In embodiments, radiation 102 may be a high-power X-Ray tube with suitable power source, a, linear accelerator (LINAC) or a synchro-cyclotron and radiation beam 104 represents all beam types as described above. A fringe of particle beam 104 passes through dosimeter 108 which monitors the beam dose and provides feedback to radiation source controller 110. In embodiments, radiation source controller 110 includes processing, memory, input/output, and communication devices as necessary to accomplish the methods described herein. In some embodiments, a portion of radiation beam 104 impacts a scintillator 130 positioned on the patient with which to monitor an entry point of the beam into the patient, the beam being aimed at a lesion or tumor 132 within the patient.

### System to Monitor High Dose Rate Output and Symmetry

In embodiments, dosimeter 108 may include a scintillator, which is a material that luminesces in response to radiation. Scintillators are typically linear across a large range of dose rates, thereby being ideal dose detectors for FLASH beam monitoring. In embodiments, a plurality of scintillator detectors is used at the beam output port of radiation source 102 and positioned so that they measure doses in the fringe or an outer region of the beam 104 that is not used for treatment; in embodiments this outer region of the beam 104 is blocked by collimator 112, the collimator allowing an inner portion of the beam as shaped beam 114. In other embodiments, dosimeter 108 may be a combination of a scintillator and photosensors or cameras (not shown) to detect light emitted by the scintillator; readings of light emitted by the scintillator are used to determine current and total radiation dose. Scintillator output is converted to dose using standard calibration mechanisms, allowing radiation source controller to determine absolute beam output and symmetry in real-time. This beam output measurement may be used in methods described herein.

### Scintillator Dosimeter Optimized for Ultra-High Dose Rate

In embodiments disclosed herein, dosimeter 108 may include an optical liquid cell filled with liquid scintillator solution, where the concentration and volume of the liquid scintillator are optimized for maximal signal and dose-rate independence while recording and reporting a time, an amplitude and a width of each individual pulse of the radiation beam. In an embodiment, the scintillator is a fluorescein solution. In another embodiment, the scintillator is a quinine solution. The optical liquid cell may be coupled to a light sensing detector (photodiode or photomultiplier tube) using an optical fiber. In an embodiment, this fiber is a hollow fiber with metal-coated walls, in order to minimize Cherenkov signal or other stray light contamination of scintillation light from the optical liquid cell. A method of dual wavelength readout may be utilized to cancel out any remaining Cherenkov signal contamination from scintillation dose.

Fringes of beam 104 passes through dosimeter 108 while the body of beam 104 enters collimator 112, which may include adjustable shielding shapes configured to determine a shape of beam 104 to form shaped beam 114. In embodiments, any type of collimator that is capable of shaping ultra-high dose rate irradiation may be used. As shown in FIG. 1, portions of beam 104 passing through dosimeter 108 are blocked by collimator 112 and thus, not used in treatment of patient 106.

In embodiments, shaped beam 114 is steered using beam scanning coil 116 before application to patient 106.

Dosimeter 108 reports time, amplitude (or intra-pulse radiation dose), and pulsewidth of detected radiation pulses of the radiation beam to dosimetry controller 111 where these pulses are integrated and scaled according to a calibration function to provide a running total of radiation dose throughout the session. In some embodiments, cameras 120 also report Cherenkov images to dosimetry controller 111 where they are processed to provide a running total of radiation dose received by the patient throughout the treatment session.

In scintillator-on-patient dosimetry embodiments, instead of, or in addition to, dosimeter 108, a semi-flexible scintillator blanket 130 is positioned at an expected beam 104 entry point on patient 106, where the scintillator blanket is in view of high-speed electronic cameras 120. This semi-flexible scintillator blanket or mask is configurable to conform largely to a surface of the patient without crinkling or folding. In alternative embodiments, flexible scintillator blanket 130 is replaced by a plastic scintillator component that in some embodiments is molded to fit on particular target portions of anatomy of a particular patient 106, for example the plastic scintillator component may be molded as a scintillator mask, scintillator skullcap, or scintillator gorget for radiation treatments of lesions 132 in a particular patient's head and neck; similarly the scintillator component may be molded as a scintillator breastplate or scintillator apron for radiation treatments of lesions in a patient's chest or abdomen. For convenience herein, the term scintillator mask is used to represent a scintillator component molded to fit any part of a patient's anatomy whether in form of mask, skullcap, breastplate, gorget, or apron. In some embodiments, fiducials for observation of patient position and/or movement are incorporated into a scintillator mask or into the semi-flexible scintillator blanket.

Scintillator materials for use in flexible scintillator blanket 130 or plastics for scintillator mask can be chosen to emit light over a wide range of beam energies, including many radiation beam energies where Cherenkov light emissions are not expected to be emitted from the patient in significant amounts.

In some embodiments using high beam energies, patient skin may be directly exposed to the radiation beam without providing a flexible scintillator blanket 130 or scintillator mask; in these embodiments, high-speed electronic cameras 120 may directly image Cherenkov light emissions from the patient.

### Dosimetry Controller

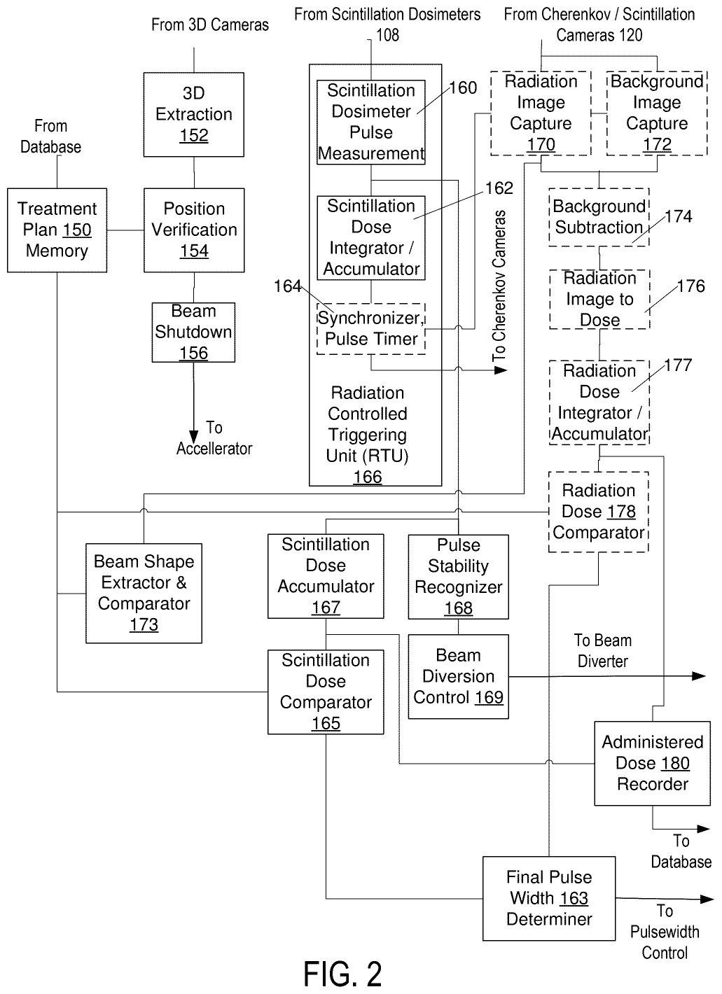

Further details of dosimetry controller 111 are shown in FIG. 2. In an embodiment, dosimetry controller 111 has a treatment plan memory 150 that is loaded from a patient database with relevant portions of a treatment plan. In addition to portions loaded into the dosimetry controller, the treatment plan also includes configuration settings for collimator 112 and for radiation source 102 that in most embodiments is a pulsed particle accelerator such as a linear accelerator, a cyclotron, or a synchrotron, or a high-power, pulsed, X-ray tube. Relevant portions of the treatment plan incorporated into treatment plan memory 150 include anticipated Cherenkov shapes and limit dosages if any, anticipated scintillation shapes from imaged scintillation blankets or masks and respective scintillation limit dosages, fiducial locations relative to patient position, expected patient position, and expected beam energy and beam shape. Locations of all cameras 120 and locations and efficiencies of all scintillation dosimeters 108 are also known to dosimetry controller 111, although these locations and efficiencies may be constant for many patients and treatment plans.

Pairs of 3D imaging cameras 120 are coupled through 3D surface extraction block 152 to position verification block 154, position verification block 154 is configured to compare extracted surfaces of patient 106 to expected patient and fiducial locations from treatment plan memory 150 and, if these differences exceed limits, beam shutdown logic 156 may be activated to discontinue or prevent radiation treatment of patient 106.

In embodiments, scintillation dosimeters 108 are coupled to a scintillation pulse measurement unit 160 of dosimetry controller 111 where time, pulse height, and width are measured for each pulse of detected scintillation, these height and width being proportional to radiation intensity and duration or width of each pulse of beam 104 and their product proportional to radiation dose administered by each pulse. These heights and widths are multiplied together and totalized in scintillation dose integrator/accumulator 162 to provide signals representing radiation dose administered by the most recent pulse and a running total of radiation dose.

In some embodiments, the time and pulse widths are fed to an optional synchronizer and pulse timer unit 164 where gating signals are prepared for cameras of cameras 120 that are capable of imaging Cherenkov radiation or of imaging scintillation blankets, the combination of scintillation pulse measurement unit 160 and synchronizer and pulse timer unit 164 being known herein as a radiation-controlled triggering unit 166.

In some embodiments, the time and pulse widths are also fed to an optional synchronizer and pulse timer unit 164 where gating signals are prepared for cameras of cameras 120 that are capable of imaging scintillation of a flexible scintillation blanket 130 or molded scintillator component, the combination of scintillation pulse measurement unit 160 and synchronizer and pulse timer unit 164 being known herein as a radiation-controlled triggering unit 166. In these embodiments, the cameras 120 are gated cameras coupled and triggered to image during radiation pulses to provide images of the scintillation blanket 130 or molded scintillator component; brightness in the images of the scintillator component at each pixel represents intensity of radiation received by the scintillator component at a corresponding point of the scintillator component and shapes of bright areas in the images represent beam shapes at beam passage through the scintillator component.

Scintillation dosimeter pulse time, height, and width are fed to a pulse stability recognizer 168 that recognizes when beam 104 pulses have become reasonably consistent in intensity and duration after radiation source 102 startup because many types of accelerators that may be used as radiation source 102 provide inconsistent pulses for a brief time after they are turned on. Until scintillation dosimeter pulses are recognized as stable by stability recognizer 168, beam diversion control 169 holds beam diverter and beam scanning coil 116 activated so shaped beam 114a is diverted into beam dump 118; after the pulses are recognized as consistent, shaped beam 114 is allowed to reach patient 106 and scintillation dose integrator/accumulator 167 is permitted to integrate and totalize radiation dose administered to patient 106.

In some embodiments, radiation dose logged by scintillation dose integrator/accumulator 167 is compared by scintillation dose comparator 165, together with dose administered by the most recent pulse of beam 104 as computed from beam pulse width and height, to planned dose information in treatment plan memory 150, when a sum of dose administered to patient 106 by the most recent pulse of beam 104 plus logged dose from scintillation dose integrator/accumulator 167 meets or exceeds planned dose, final pulse width determiner 163 computes a desired width for a final radiation pulse and provides a pulsewidth control signal to radiation source 102.

In embodiments having cameras capable of imaging Cherenkov radiation among cameras 120, or of imaging scintillator blanket 130 or from a scintillator component such as a scintillator mask, skullcap, breastplate, gorget, or apron, images from those cameras timed during pulses of shaped beam 114 are captured by radiation image capture unit 170, and background images from those cameras, timed before or significantly after but not during pulses of the shaped beam 114 are captured by background image capture unit 172. A background subtraction unit 174 then corrects radiation images for background room lighting and other stray lighting such as pilot lights and instrument lights around patient 106 into corrected radiation images. The corrected radiation images are converted to dosage maps of charge particles as administered to the patient 106 by a dose converter 176 and integrated or accumulated by radiation dose map integrator accumulator 177 before being compared to a planned dose map portion of the treatment plan in treatment plan memory 150 by a radiation dose map comparator 178. While flash treatment generally is performed in seconds to a few minutes to overwhelm DNA repair mechanisms in tissue, the pulses of the particle beam may in some embodiments be spread out over enough time that a compared dose map is ready in time for the final particle beam pulse, in these embodiments the final pulse width determiner 163 may use compared dose map in determining a width for a final pulse of shaped beam 114 to be emitted by radiation source 102. Since this width of the final pulse is computed while treatment is proceeding, and pulses are cut off according to accumulated dose, the final pulses and final pulse width are referred to as computed in real time.

Both logged dose from scintillation dose integrator/accumulator 167 and accumulated radiation dose map are recorded in a patient database by an administered dose recorder 180.

### Beam Interruption System

In standard LINACs, a large variation in beam output is acceptable over a few initial pulses, before the LINAC reaches equilibrium and becomes stable within prescribed dose-per-pulse fluctuations, typically 3-5% of nominal beam output. In FLASH therapy, this equilibrium is typically reached too late throughout the treatment, as only several pulses are planned to be delivered. In some charged-particle beam (electron or proton beam) embodiments, beam scanning coil 116 diverts shaped beam 114 as steered shaped beam 114a to beam dump 118 during an initial phase of beam output, or a ramp-up period of radiation source 102, by electromagnetically diverting shaped beam 114. Once equilibrium and beam output stability has been reached, shaped beam 114 is then steered by beam scanning coil 116 away from beam dump 118 to a targeted area on patient 106. This redirection happens between two beam pulses to avoid unintended irradiation of equipment and/or patient.

Cameras 120 are used to detect patient motion, and in some embodiments to measure and map radiation doses received by the patient by quantifying and imaging light emitted from scintillation blanket 130 or scintillation mask as the beam interacts with the scintillator, and in some embodiments Cherenkov emissions generated within and emitted from the patient as the beam interacts with tissue of the patient. In embodiments, cameras 120 may operate by imaging a variety of wavelengths. To avoid camera damage, gated electronic cameras 120, are positioned well outside shaped beam 114 with a general field of view 122 of the patient; cameras 120 are aligned such that field of view 122 includes a view of most or all of the treatment zone including a view of a surface of patient 106 that may be positioned within the treatment zone. The position of cameras 120 and their field of view 122 as shown in FIG. 1 are for purposes of illustration; more or fewer cameras may be used, and they may be placed in positions other than those shown in FIG. 1. In embodiments, cameras 120 may also include optical or infrared three-dimensional cameras 120 for tracking patient location and movement. In embodiments, cameras 120 include intensified charge-coupled device (ICCD) camera, image-intensified CMOS (ICMOS) camera or an electronically-gated, sensitive, CMOS (EGCMOS) camera, configured to image Cherenkov radiation or light from scintillator blanket 130 or a scintillator mask. In embodiments, cameras 120 may also include CMOS cameras placed in a 3-D imaging setup to detect or monitor patient position and movement.

Where cameras 120 include cameras configured to image Cherenkov radiation or light from scintillator blanket 130 or a scintillator mask as radiation images, a processor of dosimetry controller 111 is configured to use the radiation images to map radiation dose administered to the patient, to compare the mapped radiation dose administered to the patient to a treatment plan, and provide feedback to controller 110 to disable further treatment when either mapped radiation dose as administered to the patient, or integrated dosimeter 108 pulses, exceeds mapped radiation dose specifications or raw beam limitations of the treatment plan. Mapped radiation dose may also be displayed to a treating physician to determine if administered treatment was adequate to cover an entire tumor, or whether an additional treatment is required.

Cameras 120 may include several cameras each adapted to detect light from Cherenkov emissions and/or light of ultraviolet (UV), visible or near infrared (NIR) spectrums.

One or more of cameras 120 may be focused on target fiducials 124 in the region of the treatment zone. Target fiducials 124 may be artificial or biological as explained in more detail below. The locations of target fiducials 124 as shown in FIG. 1 are for purposes of illustration; more or fewer target fiducials 124 may be used, and they may be placed in positions other than those shown in FIG. 1.

Radiation source controller 110 receives feedback and/or provides control signals (not shown) to radiation source 102, dosimeter 108, collimator 12, cameras 120 and beam scanning coil 116. Other devices may also be coupled to radiation source controller 110. Connections between radiation source controller 110 may be wired or wireless.

In embodiments, the processor is configured to extract a beam shape 173 and position from the images of the scintillation blanket 130 or molded scintillator component and compare that beam shape against an expected beam shape and position from a treatment plan memory 150; if significant discrepancies are found, the image processor is configured by firmware and a connection to the radiation source to stop the radiation beam if the extracted beam shape or beam position differs by more than a threshold from a planned beam shape or position in the treatment plan memory 150.

In embodiments, the image processor is configured by firmware and coupled to the radiation source to stop the radiation beam upon the totalized radiation dosage provided by pulses of the radiation beam reaching a limit in the treatment plan memory 150.

### Real-Time Feedback Control

In embodiments, ultra-high dose rate UHDR irradiation, or FLASH therapy, delivers whole dose radiotherapy or fractions at much higher dose rates than conventional radiotherapy. For example, FLASH therapy dose rates may be above 40 Gy/s as compared to 0.01 Gy/s in a conventional radiotherapy mode. In clinical linear accelerators (LINACs), the feedback mechanisms are based on averaged readouts over extended periods of time (for example 50 ms. in Varian LINACs). This feedback mechanism is too slow for FLASH therapy dose rates.

In embodiments, system 100 uses real-time feedback control. Radiation source controller 110 includes a feedback controller that reads one or a plurality of inputs through, and running totals of radiation dose from, dosimetry controller 111; dosimetry controller 111 gets this information from various sensors including dosimeter 108 and Cherenkov cameras of cameras 120 that monitor the position and intensity of beam 104, and the treatment condition of the patient. Based on the one or plurality of inputs, radiation source controller 110 may gate an upcoming pulse from radiation source 102 off. In an embodiment, a total dose accumulator is found in dosimetry controller 111. In other embodiments, these sensors include a fast motion management system. In further embodiments, both sensors may be used in system 100.

### Pulse Modulation to Increase Absolute Dose Accuracy

Current beam monitor dosimeters are either not useful in high dose rate regimes due to strong non-linearity and/or signal to noise issues, they may experience dose-induced damage when subjected to ultra-high dose rate beams, or they are not fast enough to react on single pulse, millisecond basis.

A prescribed total radiation dose of a FLASH therapy treatment is typically delivered as a beam of a series of radiation pulses. In embodiments, approximately 2 to 100 pulses may be delivered in a FLASH therapy session. Each radiation pulse has a prescribed single pulse radiation dose less than the prescribed total radiation dose. Depending on the prescribed total radiation dose and the number of pulses used to deliver the prescribed total radiation dose, there may arise some situations in which a last pulse in the series would deliver too much radiation. This may occur, for example, if the prescribed total radiation dose is divided into a series of equal pulses, but some pulses delivered more radiation than intended due to variations within system 100.

In embodiments, the pulsewidth of an individual pulse of shaped beam 114 is modulated to change the width or intensity of a single radiation pulse. In order to maintain maximal dose rate throughout the treatment, the pulsewidth (or duration) of the last radiation pulse of the series is scaled based on a readout of dose per pulse accumulated through prior beam pulses by a total dose accumulator in dosimetry controller 111 that provides feedback to radiation source controller 110. The total dose accumulator provides an accumulated radiation dose. It may be incorporated in radiation source controller 110 or may be a separate device that communicates with radiation source controller 110. Once a difference between the prescribed total radiation dose and the accumulated radiation dose reaches a value that is smaller than a prescribed single pulse dose, a scaling factor may be applied to the width and/or intensity of the subsequent, final, pulse. In this way, overall prescribed dose accuracy increases because beam output fluctuations during treatment are accounted for.

In an embodiment, system 100 with scintillation dosimeters 108 and Cherenkov-light cameras in cameras 120 as described above is used with a beam measurement method, providing the total dose feedback data to radiation source controller 110. In another embodiment, a FLASH-capable ionization chamber-based radiation detector measures the beam output. Ionization chamber probes generally give a precise and highly localized measure of the ionizing radiation delivered by a therapeutic system at a given point, e.g., a point within a water-filled phantom. In particular embodiments, an ionization chamber is a cylindrical, waterproof Farmer-type ion ionization chamber, which is recommended by various dosimetry protocols for dose measurement of radiotherapy beams. The chambers of such probes typically have volumes of 0.6-0.65 cm3 and can report measured calibrated exposure accurate to National Institute of Standards & Technology (NIST) certified standards, which can be directly mapped to dose delivery at that point.

In an alternative embodiment, dosimetry is also performed by imaging Cherenkov radiation emitted as charged particles of the radiation beam moving at velocities less than the speed of light in a vacuum but faster than the speed of light in tissue slow to below the speed of light in tissue. In another alternative embodiment, dosimetry is also performed by imaging Cherenkov radiation emitted as high energy X-ray or gamma-ray photons kick electrons in tissue to velocities greater than the speed of light in the tissue. In these embodiments, dosimetry controller 111 is configured to use images of Cherenkov radiation emitted by tissue of the patient to map and/or totalize radiation dose administered to the patient, to compare the mapped radiation dose administered to the patient to limits of a treatment plan and provide feedback to controller 110.

In an embodiment, dosimetry is also performed by imaging scintillation light from scintillator blanket 130 or a scintillator mask that is generated when the beam interacts with said scintillator blanket 130 or scintillator mask. In these embodiments, dosimetry controller 111 is configured to use images of Cherenkov radiation emitted by tissue of the patient to map and/or totalize radiation dose administered to the patient, to compare the mapped radiation dose administered to the patient to limits of a treatment plan and provide feedback to controller 110.

Whether measured by pre-collimator scintillation dosimeters as described above, by ionization chambers as described above, by imaging Cherenkov radiation, or a combination of two or more of the three, accurate run-time dosimetry permits automatic cessation of treatment and adjustment of width or intensity of final beam pulses to avoid exceeding radiation doses specified in the treatment plan.

### Camera-Based Motion Management

In embodiments, FLASH therapy delivers the radiation dose of an entire conventional therapy session with a single fraction lasting less than a second, however, this imposes new and extreme requirements from the aspects of safety and patient positioning. In standard fractionated delivery, positioning, motion, or anatomy change errors are usually accounted for during planning and tend to average out over multiple days of treatment. However, a small deviation in patient position during FLASH therapy may have an impact on a patient's health that is orders of magnitude more severe. Anatomical shifts that may occur anytime between patient alignment and the end of beam delivery include shifts due to a non-compliant patient, inadvertent movement, and breathing motions of the patient, for example.

As shown in FIG. 1, one or several cameras 120 may be placed in locations with a view 122 of the treated area of a patient. Target fiducials 124 may be placed on the patient's skin prior the treatment to permit tracking of patient and tumor positions. Images of these fiducials are compared with planned fiducial locations in real time in order to detect motions of the patient and to gate the beam off if the detected position of the fiducials and treated area deviates from a planned treated area by more than a prescribed distance. In an embodiment, placement of target fiducial 124 may be performed as a part of treatment planning with anatomical guidance and pre-treatment X-ray computed tomography or magnetic resonance imaging images, and the cameras may observe absolute motion of the fiducials. In another embodiment, target fiducials 124 are placed immediately before treatment without anatomical guidance, and the motion is monitored relatively from the time of patient setup throughout the treatment. Deviation of the position of target fiducials 124 that is detected by cameras 120 may be provided to dosimetry controller 111 and used to control radiation source controller 110 to control radiation source 102 to gate off beam 104. Cameras 120 may work in visible and/or infrared light spectrum, the latter being useful to minimize perception by eyes. Target fiducials 124 may be reflective or fluorescent, and may be taped, tattooed, or inked onto patient skin. In some embodiments, cameras 120 configured to image fiducials are stereoscopic camera pairs and fiducial movement is observed and compared to limits in three dimensions.

In embodiments, one or more cameras 120 may work in 2D, sensitive only to motion along the imaging plane, or a plurality of cameras 120 may be used to evaluate a motion vector in 3D. The camera tracking and decision-making process happens within the time between two radiation pulses of beam 104, permitting near-instantaneous beam shutdown of excess movement is detected. In embodiments, this time may be approximately one or a few milliseconds.

The camera system as disclosed herein provides for absolute position monitoring and fast millisecond imaging and gating.

### Radiation Camera-Based Motion Management

In embodiments, cameras 120 may also include the capability of detecting and imaging Cherenkov and/or scintillation emission from tissue, or from a scintillator mask affixed to the patient. Further, the relation between the detected shape and location of the Cherenkov/scintillation area may be evaluated against the location of target fiducials 124. Biomarkers, such as the more attenuated blood vessel structures in Cherenkov/scintillation images, may in some embodiments be extracted and analyzed for motion tracking and used as additional fiducials. Beam 104 may be gated off if these relations, expressed in terms of spatial mismatch, exceed a predefined value specified in the treatment plan.

### Optimized Dose Rate Distribution Delivery Through Intensity or Width Modulation

In embodiments, beam scanning coil 116 and pulse modulation provided by total dose accumulation may be used together to optimally maximize the FLASH therapy efficacy. In particular, combining these elements of feedback and beam control provides the spatiotemporal intensity-modulation for optimizing both dose and dose rate distributions. A combination of the electromagnetic beam steering system as previously discussed with reference to beam dump 10, and the pulse modulation method as described above, enables the scanning of charged particle (electron and proton) beams with pulse-to-pulse intensity and position modulation. In embodiments, the system for ultra-high dose rate irradiation described herein may deliver dose and dose rate-optimized FLASH-RT plans through intensity-modulated scanning.

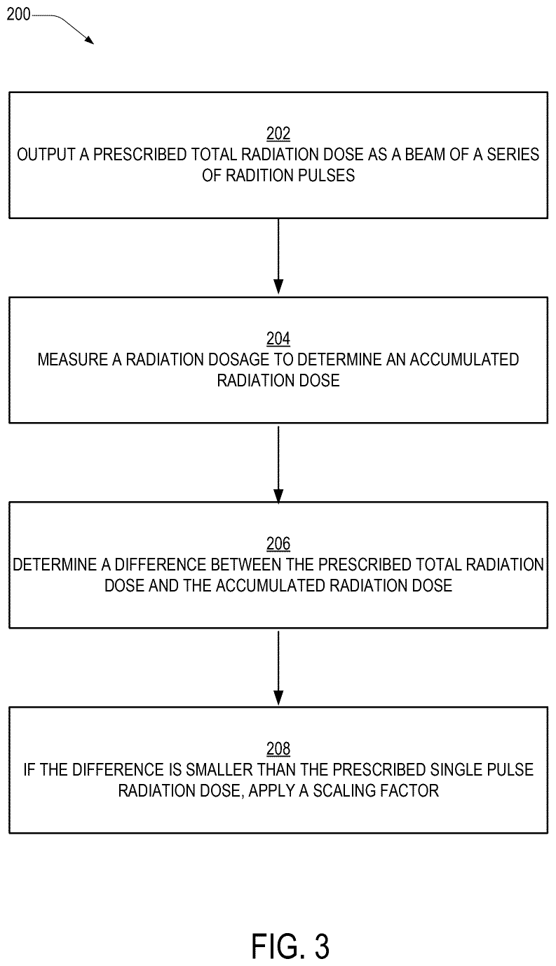

FIG. 3 depicts a flowchart of a method 200 of providing ultra-high dose rate irradiation to a target area of a patient.

Step 202 includes outputting a prescribed total radiation dose as beam of a series of radiation pulses to a target area of the patient. Each radiation pulse has a single pulse radiation dose less than the prescribed total radiation dose.

Step 204 includes measuring a radiation dose to determine an accumulated radiation dose. In an example of step 204, a radiation dose provided by each radiation pulse in the series is measured by dosimeter 108 and communicated to a total dose accumulator in dosimetry controller 110 to determine an accumulated radiation dose.

Step 206 includes determining a difference between the prescribed total radiation dose and the accumulated radiation dose. In an example of step 206, radiation pulse in the series may vary from the intended single pulse radiation dose, leading to a situation where only a partial dose should be applied in the last radiation pulse. This situation is identified by accumulating the radiation dose from each charged particle pulse and comparing the accumulated total with the prescribed total radiation dose.

Step 208 includes, if the difference is smaller than the prescribed single pulse radiation dose, applying a scaling factor to determine a desired pulse width for a final radiation pulse.

### Scintillator Blanket or Mask

In an embodiment, the scintillator blanket 130 is a semi-flexible mesh beam target. This mesh beam target includes multiple thin mesh elements interconnected via flexible links between the mesh elements. Each mesh element has a scintillator layer that can be imaged by the cameras 120. The scintillator blanket conforms to the patient's body curvature and may be attached to the patient's body with an adhesive, and upon irradiation it emits light that is collected by the camera 120 as scintillation images. The scintillation images are processed and a dose for each pulse calculated using a calibration in an image processor of dosimetry controller 111, with dose totalized over the series of pulses in each treatment session.

In embodiments, a background (e.g., room light) image of the mesh beam target 130 is captured by the cameras 120, and, using the knowledge of the mesh before deformation and knowledge of relative shifts ant tilts of the individual mesh elements in the camera's view, a 3D surface of the mesh is generated. This 3D digital mesh is then used in an angular calibration step, where the relative angle of each mesh element is used as an input parameter to the calibration routine, and an angular calibration function is used to correct the intensity of each element to minimize the effect of non-isotropic emission of the luminescence light and for viewing angles of the camera. We note that the camera views the beam target area from an angle different from that of the beam striking the beam target area. In another embodiment, the 3D surface is used to verify the correct alignment of the patient with respect to the beam isocenter.

The mesh beam target 130 is designed to be placed on the target area before beginning radiation therapy using either photon, electron, or proton irradiation. While the beam is on, the scintillator face of each element interacts with the radiation and emits visible light in direct proportion to the administered dose. The emitted light is captured using the camera 120 as images. The mesh features flexible connections that allow for consistent surface monitoring during patient cardiac, respiratory, or other small-scale movement throughout the procedure. In alternative embodiments, the mesh beam target is replaced by a scintillator mask conformed to a surface of the patient.

Once imaged by the camera, the images of the mesh are corrected to provide a surface dose map for each pulse of the beam. Each surface dose map is corrected for angular emission dependence and local surface abnormalities to provide reliable data on expected surface dose. This correction may include but is not limited to factors such as distance, dose rate, linearity, perspective, and source-camera angle.

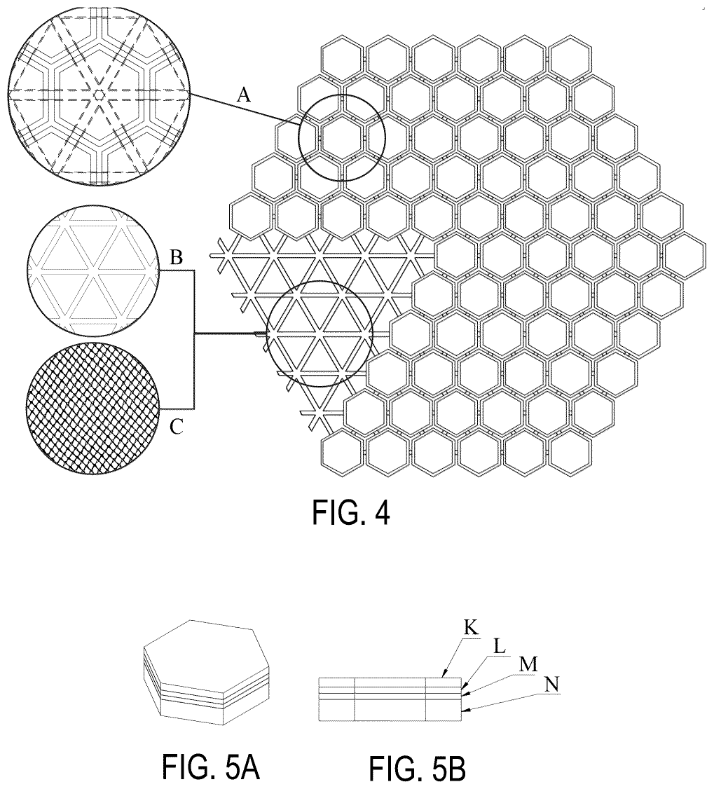

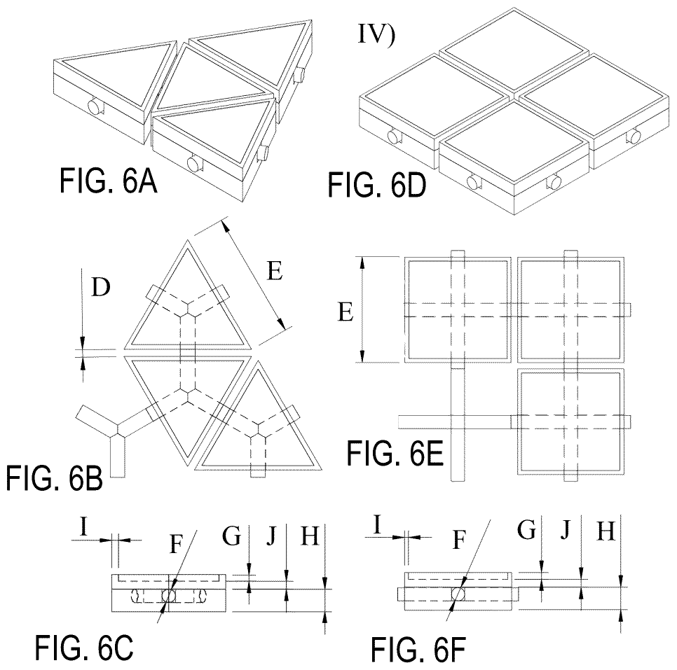

In embodiments, the mesh beam target 130 has hexagonal shaped elements as illustrated in FIGS. 4, 5A, and 5B. In alternative embodiments the mesh beam target has triangular shaped elements as illustrated in FIGS. 6A, 6B, and 6C, and square elements as illustrated in FIGS. 6D, 6E, and 6F. Other shapes may be used including custom shaped elements. The elements are connected by a mesh of string, or flexible filament (designated as “B”) with one connection between each successive element, or a fine mesh (designated as “C”) with several connections between successive elements. The top layer of each element includes a scintillator layer (K) nestled and secured on top surface of the mesh element; additional layers L, M, and N have structural and element-linking functions. In specific embodiments, this scintillator is seated within an opaque holder of the plaque featuring optional ridges along the edges to prevent optical light piping during irradiation. The bottom layer of the mesh element matches the shape of the top and is constructed from high-density plastic to provide structural support and add weight to aid deformation. In an embodiment which utilizes the mesh connector design (B), the backing layer includes channels to house individual strands. The sizes and relative distances of each component are detailed in the feature list:
a. ultrafast gated camera with pulsed LED to reject background light
b. arterial input function acquisition with pulse dye densitometry
c. tracer kinetic modeling using the adiabatic approximation to the tissue homogeneity (AATH) model, the hybrid plug-compartment (HyPC) model, and/or non-parametric deconvolution models such as constrained or regularized (e.g., truncated SVD, Tikhonov SVD, etc.).
d. extraction of spatial, temporal and dynamic (kinetic model) features, and their use in machine learning models.
e. predicts and displays the “tissue sparing risk”—i.e., the risk that sparing that tissue will have on patient outcome (similar to a false negative rate, but connects pixel-level data to patient-level outcome).
f. live feedback to surgeon regarding pixel-level classification providing actionable information during debridement surgery.

In a particular embodiment, the mesh beam target 130 is manufactured using 3D printing for the backing and upper housing components, using PLA material. If the large mesh design is utilized, a dual extruder printer and flexible filament will be used to make the connections. For the fine mesh design, the backing print will be paused after 2-3 layers, the polyester mesh will be stretched across the build plate, secured, and the print will be continued with a slight vertical offset to integrate the mesh into the build. In both designs, scintillator components will be inserted and secured into their respective housing. In another embodiment, the mesh beam target 130 is created by 3D polyjet printing, where the mesh elements are composed of a black opaque material and interconnecting elements are printed with a flexible material.

In embodiments: the scintillator mesh target has four portions: a thin scintillator on top and visible to the cameras 120, thin scintillator housing, flexible interconnecting mesh, and a clinical adhesive layer.
1. The scintillator is cut from a sheet of highly uniform stock layer of <1 mm thick sheet of a powdered scintillator embedded in plastic matrix, or of a plastic scintillator. This ensures homogeneity of scintillation response. Hexagons or triangle shaped scintillator elements maximize the number of deformation angles.
2. The housing is a thin plastic (tissue equivalent) cell that supports the scintillator; it is slightly oversized than the scintillator and walls are raised around the scintillator, to prevent imaging side emission, and to limit crosstalk.
3. The mesh is currently a thin plastic fabric that is embedded by 3D printing the mesh in 2 stages; first, a thin supporting layer is printed; then a flexible fabric is laid over the print; and 3rd, top layer of the mesh is printed over the fabric. The fabric has holes large enough to allow fusing the bottom and top plastic layers. In alternative embodiments, the scintillator plaques may be formed separately from the mesh and then attached to the mesh.

Once the 3D plastic mesh is printed, the scintillators are glued to each cell scintillator side up.

In a particular embodiment, the scintillator mesh uses Scintacor (Cambridge, UK) Rapidex scintillators in combination with a high speed camera having a fast CMOS sensor (Luxima (Arcadia, California) LUX19HS, >12000 fps) and an image intensifier with P46 phosphor—this is to accurately image dose rate maps of ultra-high dose rate scanned pencil proton beams. The overall temporal response is preferably less than 1 millisecond to prevent temporal smearing and to maintain accuracy of the PBS dose rates.

In an alternative embodiment for lower beam intensities, the scintillator mesh target is a flexible cesium iodide scintillator such as is available from Scintacor on a polyester flexible backing.

In an alternative embodiment the scintillator mask is formed of a plastic scintillator having the embedded lanthanide scintillator powder as previously described. In an alternative embodiment Scintacor (Cambridge, UK) NDFast material with 3.5 us decay time using a scintillation compound that includes a lithium 6 fluoride-zinc oxide-zinc (LiF ZnO:Zn) scintillator having a ZnO:Li6 ratio between 2:1 and 4:1 is used, this NDFast material is heat formable on polyester substrate and has peak emissions at 500 nm.

To prevent spatio-temporal blur due to a finite luminescence decay of the luminescence layer, the scintillator should have a short decay time. In embodiments the scintillator is a lanthanide rare earth scintillator selected from Gadolinium oxide GaO2S:Pr (prascodymium) or GaO2S:Tb (terbium); with GaO2S:Pr having a shorter decay time and preferred for FLASH therapy Both lanthanide rare earths have short characteristic decay time under 1 millisecond. In embodiments, the scintillator layer comprises Gd2O2S:Pr material which has typically 7 microsecond decay time to 10% intensity.

To prevent spatio-temporal blur due to a long temporal response of the image intensifier, the phosphor at the intensifier output window may have a less than one ms. characteristic decay time. In embodiments, the camera 120 is an intensified CMOS camera wherein the phosphor material in the intensifier is of P46 type; In other embodiments, the camera 120 is an intensified CMOS camera wherein the phosphor material in the intensifier is of P47 type;

In some embodiments, cameras 120 include at least two cameras having different filters, wherein the filters are chosen at two different wavelength bands, and the relative difference of the images these cameras produce are used to distinguish Cherenkov from scintillation light.

In an alternative embodiment, the beam is quasi-continuous as it is pulsed at a rate of tens to hundreds of megahertz, it is impractical to record dose pulse-by-pulse with a quasi-continuous beam. In particular, some scanned proton beams are of the quasi-continuous type. With quasi-continuous beam embodiments, images are captured by cameras 120 at a high, but practical, rate typically exceeding 30 frames per second and in a particular embodiment 4000 frames per second. Once captured, these images are processed as described above for images captured with each pulse of pulsed radiation beams to provide a running cumulative dose image. As described above, when the cumulative dose image is found to be out of limits or to exceed planned dosage, the controller 111 shuts off the beam.

In alternative embodiments, the camera is a non-intensified single-photon avalanche photodiode (SPAD) camera or a non-intensified, high-sensitivity, CMOS camera; these embodiments may be particularly useful for monitoring non-UHDR or standard radiation treatment systems.

In embodiments where cameras 120 include 3D imaging capability, The 3D information may be used to geometrically transform the captured scintillation-based dose map and dose rate map from point of view of the camera to a beams-eye view. This allows direct comparison of the measured dose and dose-rate map with respect to planned dose/dose rate maps, and automatic stopping of the beam when the measured dose and/or dose-rate map differ significantly from a planned dose/dose-rate map. This geometric translation or projection onto a plane perpendicular to the beam axis is the most optimal way to perform quality assurance of delivered beams, because the treatment plan is usually translated to one or more beam distributions in a coordinate plane that is perpendicular to the beam's central axis allowing direct comparison.

In some embodiments, an additional scintillator may be positioned in the beam, and the second scintillator is imaged by another camera, to provide a second reference point in the beam defining a beam line.

In some embodiments, before positioning a scintillator blanket or mask on a subject for radiation treatment, the scintillator is positioned in a beam from the pulsed radiation source and an ionization detector is positioned in the beam. The beam is run briefly and imaged by the camera 120 to generate calibration images while the ionization detector measures the beam. A calibration equation is derived between images of the scintillator and dose measured by the ionization detector. Once calibrated, the scintillator is positioned on a subject for radiation treatment and treatment begun as previously described.

### Combinations

The various ideas included in this document can be combined in various ways. Among the ways anticipated by the inventors are:

A system designated A for delivering a radiation beam selected from an electron beam, a proton beam, a heavier charged particle beam, and an X-ray beam to a target area of a patient, includes a radiation source for providing a beam of radiation in a series of pulses along a beam axis; at least one controller for providing control signals to the radiation source; a scintillator selected from a scintillator blanket and a scintillator mask, the scintillator conformable to a surface of the patient in the target area of the patient; and at least one camera configured to prepare images of the scintillator on the target area of the patient from a distance. The system also includes an image processor coupled to receive the images of the scintillator from the at least one camera, the image processor configured to use the images of the scintillator to measure a radiation dosage provided by each pulse of the radiation beam, to totalize the radiation dosage provided by pulses of the radiation beam, and to provide signals to the at least one controller.

A system designated AA including the system designated A wherein the image processor is configured by firmware in memory to extract an extracted beam shape or an extracted beam position from the images of the scintillator.

A system designated AB including the system designated AA wherein the image processor is configured by firmware and a connection to the radiation source to stop the radiation beam if the extracted beam shape differs by more than a threshold from a planned beam shape.

A system designated AC including the system designated AA wherein the image processor is configured by firmware and a connection to the radiation source to stop the radiation beam if the extracted beam position differs by more than a threshold from a planned beam position.

A system designated AD including the system designated A, AA, AB, or AC wherein the image processor is configured by firmware and coupled to the radiation source to stop the radiation beam upon a totalized radiation dosage provided by pulses of the radiation beam reaching a limit in a treatment plan.

A system designated AE including the system designated A, AA, AB, AC, or AD wherein the scintillator comprises a lithium 6 fluoride-zinc oxide-zinc (LiF ZnO:Zn) scintillation compound having a ZnO:Li6 ratio between 2:1 and 4:1.

A system designated AF including the system designated A, AA, AB, AC, AD, or AE wherein the at least one camera is a high speed gated camera coupled to image during pulses of the beam of radiation.

A system designated AG including the system designated A, AA, AB, AC, AD, AE, or AF wherein a second scintillator is provided in the radiation beam and the second scintillator is imaged by a camera to provide a beam line.

A method designated B of monitoring radiation treatment of a patient includes placing a scintillator selected from a scintillator blanket or a scintillator mask on a target area of the patient; providing a radiation beam through the scintillator into the target area of the patient; observing light emitted by the scintillator with at least one camera to provide scintillation images of the scintillator while providing the radiation beam, the at least one camera positioned outside the radiation beam and positioned to image the scintillator from a distance; and applying dose calibration factors, angular emission correction factors, and a measured 3D surface of the scintillator, to generate maps of dose and dose rate. In particular embodiments, the maps of dose and/or maps of dose rate are prepared as projections onto a geometric plane perpendicular to an axis of the radiation beam.

A method designated BA including the method designated B further including extracting an extracted beam shape or an extracted beam position from the images of the scintillator.

A method designated BB including the method designated BA further including comparing the extracted beam shape to a planned beam shape and stopping the radiation beam if the extracted beam shape differs by more than a threshold from the planned beam shape.

A method designated BC including the method designated BA further including stopping the radiation beam if the extracted beam position differs by more than a threshold from a planned beam position.

A method designated BD including the method designated B, BA, BB, or BC further including stopping the radiation beam upon a totalized radiation dosage provided by pulses of the radiation beam reaching a limit in a treatment plan.

A method designated BE including the method designated B, BA, BB, BC, or BD wherein the scintillator comprises a lithium 6 fluoride-zinc oxide-zinc (LiF ZnO:Zn) scintillation compound having a ZnO:Li6 ratio between 2:1 and 4:1.

A method designated BF including the method designated B, BA, BB, BC, BD, or BE wherein the at least one camera is a high speed gated camera coupled to image during pulses of the radiation beam.

A method designated BG including the method designated B, BA, BB, BC, BD, BE, or BF further including imaging a second scintillator in the radiation beam to provide a beam line.

Changes may be made in the above methods and systems without departing from the scope hereof. It should thus be noted that the matter contained in the above description or shown in the accompanying drawings should be interpreted as illustrative and not in a limiting sense. Herein, and unless otherwise indicated: (a) the adjective “exemplary” means serving as an example, instance, or illustration, and (b) the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

What is claimed is:

1. A system for delivering a radiation beam selected from an electron beam, a proton beam, a heavier charged particle beam, and an X-ray beam to a target area of a patient, comprising:
a radiation source for providing a beam of radiation in a series of pulses along a beam axis;
at least one controller for providing control signals to the radiation source;
a scintillator selected from a scintillator blanket and a scintillator mask, the scintillator conformable to a surface of the patient in the target area of the patient;
at least one camera configured to prepare images of the scintillator on the target area of the patient from a distance; and
an image processor coupled to receive the images of the scintillator from the at least one camera, the image processor configured to use the images of the scintillator to measure a radiation dosage provided by each pulse of the radiation beam, to totalize the radiation dosage provided by pulses of the radiation beam, and to provide signals to the at least one controller.

2. The system of claim 1 wherein the image processor is configured by firmware in memory to extract an extracted beam shape or an extracted beam position from the images of the scintillator.

3. The system of claim 2 wherein the image processor is configured by firmware and a connection to the radiation source to stop the radiation beam if the extracted beam shape differs by more than a threshold from a planned beam shape.

4. The system of claim 2 wherein the image processor is configured by firmware and a connection to the radiation source to stop the radiation beam if the extracted beam position differs by more than a threshold from a planned beam position.

5. The system of claim 1 wherein the image processor is configured by firmware and coupled to the radiation source to stop the radiation beam upon a totalized radiation dosage provided by pulses of the radiation beam reaching a limit in a treatment plan.

6. The system of claim 1 wherein the scintillator comprises a lithium 6 fluoride-zinc oxide-zinc (LiF ZnO:Zn) scintillation compound having a ZnO:Li6 ratio between 2:1 and 4:1.

7. The system of claim 1 wherein the at least one camera is a high speed gated camera coupled to image during pulses of the beam of radiation.

8. The system of claim 1 wherein a second scintillator is provided in the radiation beam and the second scintillator is imaged by a camera to provide a beam line.

9. The system of claim 1 wherein the image processor is configured to generate maps of dose and dose rate.

10. The system of claim 9 wherein the image processor is configured to project the maps of dose into a geometric plane perpendicular to an axis of the radiation beam.

11. A method of monitoring radiation treatment of a patient comprising:
placing a scintillator selected from a scintillator blanket or a scintillator mask on a target area of the patient;
providing a radiation beam through the scintillator into the target area of the patient;
observing light emitted by the scintillator with at least one camera to provide scintillation images of the scintillator while providing the radiation beam, the at least one camera positioned outside the radiation beam and positioned to image the scintillator from a distance; and
applying dose calibration factors, angular emission correction factors, and a measured 3D surface of the scintillator, to generate maps of dose and dose rate.

12. The method of claim 11 further comprising extracting an extracted beam shape or an extracted beam position from the images of the scintillator.

13. The method of claim 12 further comprising comparing the extracted beam shape to a planned beam shape and stopping the radiation beam if the extracted beam shape differs by more than a threshold from the planned beam shape.

14. The method of claim 12 further comprising stopping the radiation beam if the extracted beam position differs by more than a threshold from a planned beam position.

15. The method of claim 12 further comprising stopping the radiation beam upon a totalized radiation dosage provided by pulses of the radiation beam reaching a limit in a treatment plan.

16. The method of claim 11 wherein the scintillator comprises a lithium 6 fluoride-zinc oxide-zinc (LiF ZnO:Zn) scintillation compound having a ZnO:Li6 ratio between 2:1 and 4:1.

17. The method of claim 11 wherein the at least one camera is a high speed gated camera coupled to image during pulses of the radiation beam.

18. The method of claim 11 further comprising imaging a second scintillator in the radiation beam to provide a beam line.

19. The method of claim 11 wherein the maps of dose and dose rate are projected into a geometric plane perpendicular to an axis of the radiation beam.
